(19) United States
(12) Patent Application Publication
(10) Pub. No.: US 2013/0178703 A1
(43) Pub. Date: Jul. 11, 2013

(54) Endoscope Shaft With Malleable Section

(71) Applicant: Gregory S. Konstorum, Stamford, CT (US)

(72) Inventor: Gregory S. Konstorum, Stamford, CT (US)

(73) Assignee: Gyrus ACMI, Inc.

(21) Appl. No.: 13/344,078

(22) Filed: Jan. 5, 2012

(51) Int. Cl. A61B 1/00

(52) U.S. Cl. 600/114

(57) ABSTRACT

An endoscope including a control section; and a shaft extending from the control section. The shaft includes a malleable section and a distal active deflection section. The shaft includes a frame and at least one active deflection control wire extending through the malleable section between the control section and the distal active deflection section. The frame includes a malleable frame member at the malleable section. The malleable frame member connects a frame member of the frame at the distal active deflection section to the control section.

### BACKGROUND

1. Technical Field

The exemplary and non-limiting embodiments relate generally to an endoscope and, more particularly, to an endoscope shaft.

2. Brief Description of Prior Developments

U.S. Pat. No. 7,559,925 discloses a malleable sinus guide used to insert a shaft of an endoscope. U.S. Pat. No. 5,876,330 discloses a semi-rigid shaft with a malleable tip.

### SUMMARY

The following summary is merely intended to be exemplary. The summary is not intended to limit the scope of the claims.

In accordance with one aspect, an endoscope is provided including a control section; and a shaft extending from the control section. The shaft includes a malleable section and a distal active deflection section. The shaft includes a frame and at least one active deflection control wire extending through the malleable section between the control section and the distal active deflection section. The frame includes a malleable frame member at the malleable section. The malleable frame member connects a frame member of the frame at the distal active deflection section to the control section.

In accordance with another aspect, a method comprises bending a malleable section of a shaft of an endoscope, where the shaft comprises an active deflection section located at a distal end of the shaft, where the active deflection section is connected by the malleable section to a control section of the endoscope, and where the active deflection section of the shaft is relocated when the malleable section is bent; and deflecting the active deflection section to move the distal end of the endoscope shaft relative to the malleable section.

In accordance with another aspect, a method comprises connecting a frame member of an active deflection section of an endoscope shaft to a malleable frame member of the endoscope shaft, where the malleable frame member is part of a malleable deflectable section of the endoscope shaft; and connecting the malleable frame member to a control section of an endoscope, where the malleable frame member connects the frame member of the active deflection section to the control section.

### BRIEF DESCRIPTION OF THE DRAWINGS

The foregoing aspects and other features are explained in the following description, taken in connection with the accompanying drawings, wherein:

FIG. 1 is a perspective view of an example embodiment of an instrument;

FIG. 2 is a side view of the shaft of the instrument shown in FIG. 1 with the malleable section in a straight postion;

FIG. 3 is a side view of the shaft as in FIG. 2 with the malleable section in a bent position;

FIG. 4 is a partial enlarged view of frame members of the shaft shown in FIGS. 1-3;

FIG. 4A is a sectional view taken along line 4A-4A in FIG. 4;

FIG. 4B is a sectional view taken along line 4B-4B in FIG. 4;

FIG. 5 is a diagram illustrating a removable connection of the malleable frame member shown in FIG. 4 to another frame member; and

FIG. 6 is a partial cross sectional view of an alternate embodiment of the shaft frame.

### DETAILED DESCRIPTION OF EMBODIMENTS

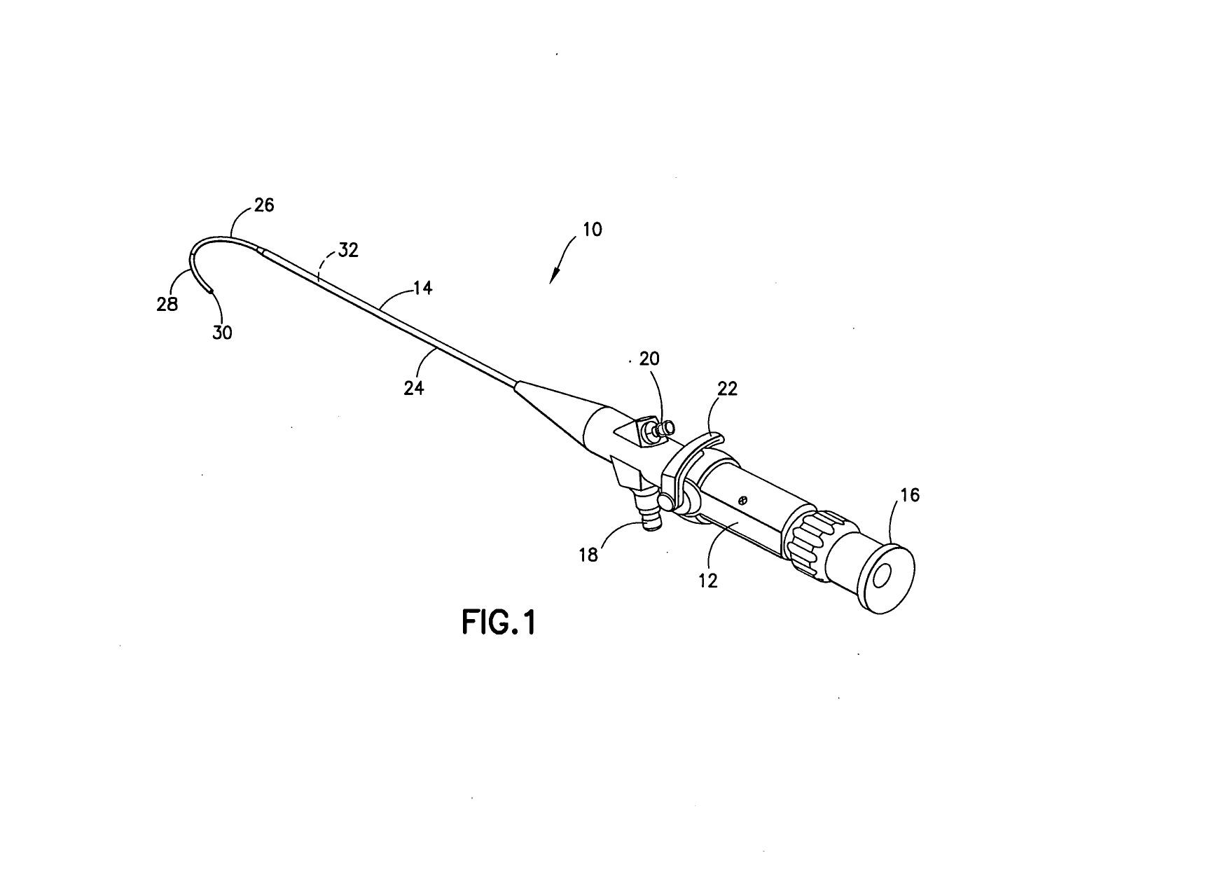

Referring to FIG. 1, there is shown a perspective view of an instrument 10 incorporating features of an example embodiment. Although the features will be described with reference to the example embodiments shown in the drawings, it should be understood that features can be embodied in many alternate forms of embodiments. In addition, any suitable size, shape or type of elements or materials could be used.

The instrument 10 is an endoscope comprising a control section 12 and a shaft 14. The control section 12 is a conventional control section forming a handle for the endoscope. In the example embodiment shown, the control section 12 includes an eyepiece 16, a light post 18, a working channel port 20, and a deflection control 22 lever. However, in alternate embodiments any suitable type of control section could be provided.

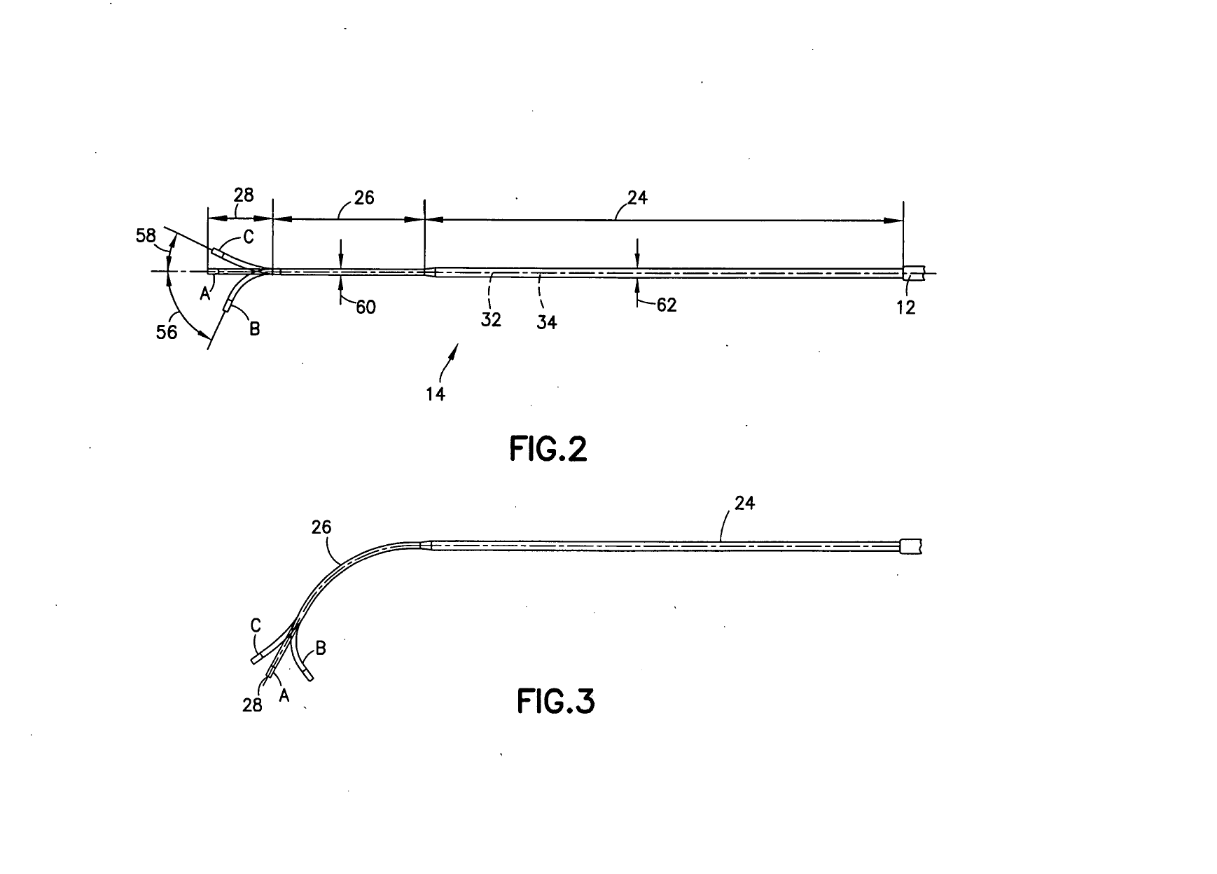

Referring also to FIG. 2, the shaft 14 extends from the control section 12 in a general cantilever fashion. The shaft 14 includes a rigid section 24, a malleable section 26, and a distal active deflection section 28. The end of the active deflection section 28 has an objective head 30. The end of the active deflection section 28 also has an aperture for the working channel 32 from the working channel port 20. The shaft 14 includes a frame, the working channel 32, fiber optics or electrical wires to the optics in the distal tip, such as for illumination and/or visualization, and deflection control wires between the deflection control lever 22 and the frame of the active deflection section 28.

FIG. 2 shows the malleable section 26 in a straight position. Position A shows the active deflection section 28 also in a straight, home position. The user can move the lever 22 to thereby move the deflection control wires 34 and deflect the active deflection section 28 into different positions, such as position B and position C shown in FIG. 2. In the example shown, the active deflection section is two way deflectable; angle 56 down and angle 58 up from the straight home positions A. In the example shown, angle 56 is about 110 degrees and angle 58 is about 35 degrees. However, in alternate embodiments the angles could be more or less, and the deflections could be more or less than two way deflectable from the home position.

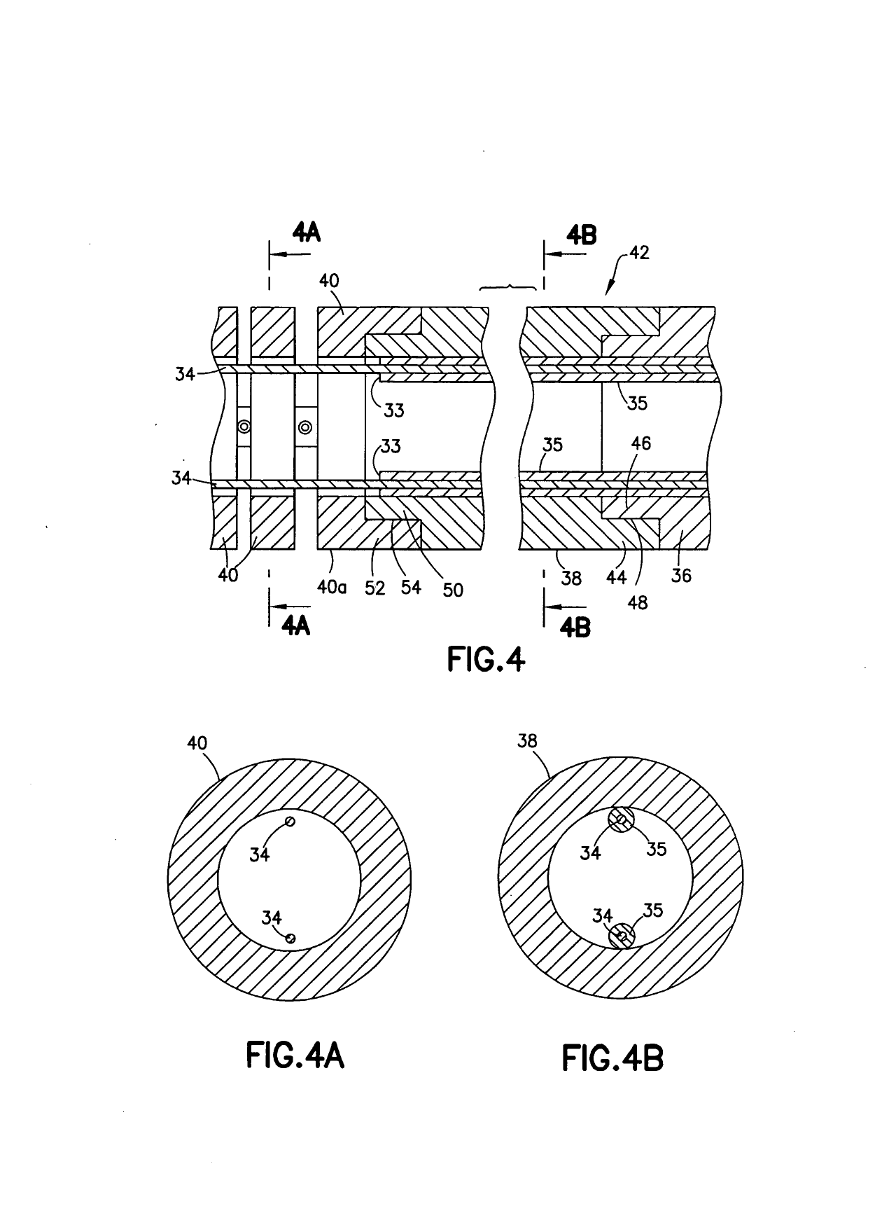

Referring also to FIG. 4, the frame 42 of the shaft 14 comprises a substantially rigid tube 36 at the rigid section 24, such as stainless steel for example. The frame 42 at the malleable section 26 comprises a tube comprised of malleable material such as metal for example. The malleable frame member 38 could be comprises of a plurality of materials, such as including a superelastic shape memory material for example. The frame 42 at the active deflection section 28 comprises a plurality of rings 40 pivotably connected to each other. In an alternate embodiment the frame at the active deflection section 28 could comprise a tube with slots such as described in U.S. Pat. Nos. 6,749,560 and 6,780,151 which are hereby incorporated by reference in their entireties. However, any suitable frame construction could be provided in the active deflection section 28.

In this example, the proximal end 44 of the malleable frame member 38 is fixedly, stationarily connected to the distal end 46 of the rigid frame member 36 at a connection 48. In addition, the distal end 50 of the malleable frame member 38 is fixedly, stationarily connected to the proximal end 52 of the frame member 40a at a connection 54. Any suitable type of connection could be provided for the fixedly, stationary connections 48, 54. The malleable frame member 38 structurally connects the frame rings 40 of the frame at the active deflection section 28 to the rigid frame member 36 which is connected to the frame of the control section 12. One or more cover members is provided over the frame 42.

Referring also to FIG. 3, the malleable frame member 38 allows the user to bend the malleable section 26 into a curved shape, and the malleable frame member 38 is then able to retain that bent shape during use of the instrument inside the patient. The rigid section 24 remains straight and provides sufficient column strength and torque stability to move the malleable section 26 and active deflection section 28 through the sinus cavities to a desired location or along a desired path in the sinus cavities.

As seen in FIG. 3, the malleable section 26 can be deformed or bent relative to the rigid section 24. This relocates the active deflection section 28 relative to the rigid section 24. The active deflection section 28 is still able to deflect among the various positions A-B-C even though it has been relocated.

Due to the very limited space and anatomy specifics for human sinus procedures, the sinus endoscope shaft distal portion outside diameter 60 should be not more than 1.8 mm, and it should have adequate column strength and torque stability. Because of these limitations a rigid scope cannot be inserted into the maxillary or frontal sinuses. A flexible scope of this size does not have adequate column strength that makes it difficult to push through the nasal passage. An endoscope with a rigid shaft and a steerable distal portion also has some limitation for this procedure. The features described above can be provided in a sinus endoscope which includes an endoscope shaft having an active deflection section and a malleable shaft section.

The example endoscope design has a rigid shaft, a steerable distal portion and a malleable section between them. The rigid shaft could have an outside diameter 62 of up to about a 3 mm for example. The steerable distal portion and malleable section could each have an outside diameter of up to 1.8 mm for example. This example has sufficient column strength and torque stability for a sinus endoscope. The malleable section can be pre-bent by a doctor in a required direction and shaped based upon the specific sinus anatomy of the patient.

The endoscope shaft example dimensions noted above can provide particularly good results based on human sinus anatomy. In particular, a sinus endoscope with a shaft having a 3 mm diameter rigid shaft, malleable and active deflection sections with 1.8 mm outside diameter, where active deflection can be deflected to +110°/−35° angles, is particularly advantageous.

An example embodiment may be provided in an endoscope 10 comprising a control section 12; and a shaft extending from the control section. The shaft may comprise a malleable section 26 and a distal active deflection section 28. The shaft comprises a frame 42 and at least one active deflection control wire 34 extending through the malleable section 26 between the control section 12 and the distal active deflection section 28. The frame 42 comprises a malleable frame member 38 at the malleable section, where the malleable frame member connects a frame member 40a of the frame at the distal active deflection section 28 to the control section 12.

The malleable section 26 may have an outside diameter of about 1.8 mm or less, and the rigid frame member 36 or rigid section 24 may have an outside diameter of about 3 mm or less in order to comfortably insertable into a sinus cavity of a patient.

The frame 42 of the shaft may further comprise a rigid frame member 36 connecting the malleable frame member 38 to the control section 12. The malleable frame member 38 may be removably connected rigid frame member 36. The malleable section 26 may be adapted to be manually bent into a curved configuration, where the distal active deflection section 28 is configured to be bent by the at least one active deflection control wire 34 without substantially movement of the malleable frame member 38, where the malleable frame member 38 retains the malleable section in the curved configuration while the distal active deflection section is bent by the at least one active deflection control wire. The control section 12 may comprise a user actuated deflection control lever 22 connected to the at least one active deflection control wire 34. The distal active deflection section 28 may comprise a plurality of the frame members at the distal active deflection section pivotably connected to each other. The frame member at the distal active deflection section may comprise a tube shape with a plurality of slots therein. The malleable frame member may comprise a metal tube. The malleable frame member may comprise a super-elastic shape memory material.

Referring also to FIGS. 4A and 4B, in this example the instrument comprises two of the deflection control wires 34. However, in alternate embodiments more or less than two deflection control wires could be provided. In the example shown, the instrument comprises cable sheaths 35. In this example the distal ends 33 of the cable sheaths 35 terminate in the malleable frame member 38. In particular, in this example the distal ends 33 are fixedly, permanently, stationarily connected to the malleable frame member 38 near the connection 54. In an alternate example the cable sheaths 35 could be removably attached to the malleable frame member 38. In one type of an alternate embodiment the distal ends 33 of the cable sheaths 35 could terminate in one of the active deflection section frame members 40. The cable sheaths 35 could be connected to the malleable frame member 38 at more than one location along the length of the malleable frame member 38; not necessarily just near the connection 54. In the example embodiment shown, the deflection control wires 34 travel through the frame members 40 without the Sheaths 35 covering the wires. However, in an alternate embodiment the sheaths 35 could cover the wires 34 along at least some of the length of the active deflections section.

For comfortably insertion of the shaft into a sinus cavity of a patient, the length of the rigid section 24 may be about 160 mm for example, the length of the malleable section 26 may be about 50 mm for example, and the length of the active deflection section 28 may be about 22 mm for example. These lengths, the outer diameters and angles of deflection of the active deflection section, and column strength and torque stability provided by the frame members 36, 38 provide easier insertion of the shaft into the sinus cavity, and faster viewing of areas inside the sinus cavity because the doctor can get the objective head to the desired sinus cavity area faster than with conventional instruments.

An example method may comprise bending a malleable section 26 of a shaft 14 of an endoscope, where the shaft comprises an active deflection section 28 located at a distal end of the shaft, where the active deflection section is connected by the malleable section to a control section 12 of the endoscope, and where the active deflection section of the shaft is relocated when the malleable section 26 is bent; and deflecting the active deflection section 28 to move the distal end of the endoscope shaft relative to the malleable section.

Deflecting the active deflection section may comprise the control section being used to deflect the active deflection section, and where the malleable section is configured to substantially retain its shape and not be moved while the active deflection section is being deflected by the control section. The malleable section may be connected to the control section by an elongate rigid frame member, where bending the malleable section comprises the malleable section being bent relative to the elongate rigid frame member.

Another example method may comprise connecting a frame member 40a of an active deflection section 28 of an endoscope shaft to a malleable frame member 38 of the endoscope shaft, where the malleable frame member is part of a malleable deflectable section 26 of the endoscope shaft; and connecting the malleable frame member to a control section 12 of an endoscope, where the malleable frame member connects the frame member of the active deflection section to the control section. Connecting the malleable frame member to the control section may comprise connecting an elongate rigid frame member 36 to the control section and connecting a rear end of the malleable frame member to a front end of the elongate rigid frame member. Connecting the malleable frame member to a front end of the elongate rigid frame member may comprise removably connecting the malleable frame member to the rigid frame member. Connecting the malleable frame member to a front end of the elongate rigid frame member may comprise permanently connecting the malleable frame member to the rigid frame member. Connecting the frame member of the active deflection section to the malleable frame member may comprises permanently connecting the malleable frame member to the frame member of the active deflection section. The method may further comprise locating at least one active deflection control wire 34 through the malleable frame member 38 from the control section to the active deflection section.

Referring also to FIG. 5, the connections 48 and/or 54 may be removable connections, such as the threaded connection 64. This allows the tube 38 to be replaced to thereby extend the service life of the instrument 10.

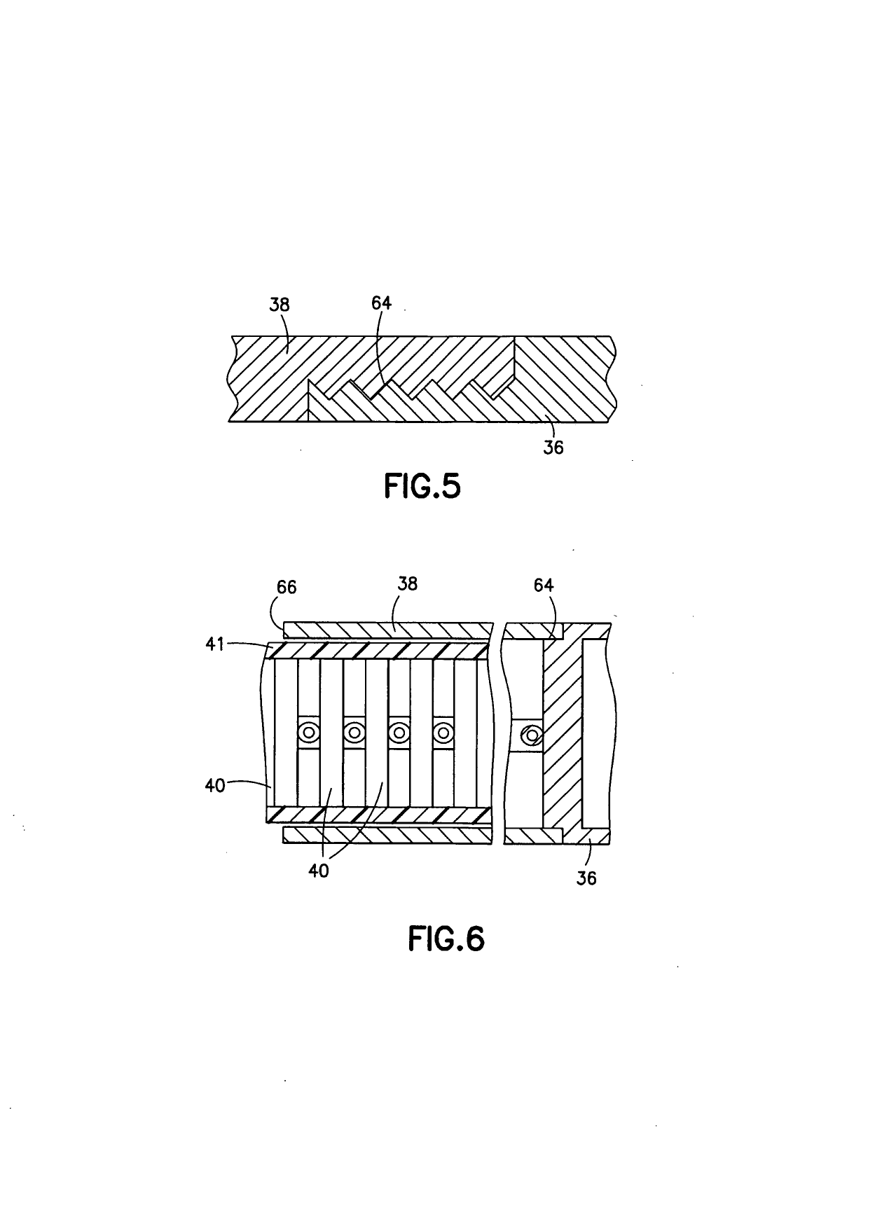

Referring also to FIG. 6, an alternate embodiment is shown. In this example the frame 40 of the active deflection section 28 could be connected to the rigid frame member 36, and the malleable frame member 38 could extend over some of the rings 40 and their cover 41. The malleable frame member 38 is shown extending from the rigid frame member 36 in a general cantilever fashion with a free end 66. With the removable connection 64, the malleable frame member is more easily replaced, but an still provide a malleable frame section in conjunction with the some of the rings 40.

In one example embodiment the malleable section is stiffer than the active deflection section. Thus, the malleable section is able to keep its bent shape while the active deflection section is being moved by the deflection control wires 34. However, in one type of alternate embodiment the shaft may be designed such that the malleable section deflects slightly when the active deflection section is moved. In one type of alternate embodiment the stiffness of at least a portion of the malleable section could be less stiff than the active deflection section. The wall thickness of the malleable frame member 38 could be non-uniform along its length to provide different malleable stiffness properties at different locations along the length.

With one example, the endoscope may comprise at least one cable sheath 35 covering at least one active deflection control wire 34, where the at least one active deflection control wire 34 extends through the active deflection section 28 without being covered by the at least one cable sheath 35. The at least one cable sheath may have a distal end 33 which ends in the malleable frame member 38. The at least one cable sheath 35 may be directly fixedly connected to the malleable frame member.

It should be understood that the foregoing description is only illustrative. Various alternatives and modifications can be devised by those skilled in the art. For example, features recited in the various dependent claims could be combined with each other in any suitable combination(s). In addition, features from different embodiments described above could be selectively combined into a new embodiment. Accordingly, the description is intended to embrace all such alternatives, modifications and variances which fall within the scope of the appended claims.

What is claimed is:

1. An endoscope comprising:
a control section; and
a shaft extending from the control section, where the shaft comprises a malleable section and a distal active deflection section, where the shaft comprises a frame and at least one active deflection control wire extending through the malleable section between the control section and the distal active deflection section, and where the frame comprises a malleable frame member at the malleable section, where the malleable frame member connects a frame member of the frame at the distal active deflection section to the control section.

2. An endoscope as in claim 1 where the frame of the shaft further comprises a rigid frame member connecting the malleable frame member to the control section.

3. An endoscope as in claim 2 where the malleable section has an outside diameter of about 1.8 mm or less, and where the rigid frame member comprises an outside diameter of about 3 mm or less in order to comfortably insertable into a sinus cavity of a patient.

4. An endoscope as in claim 3 where a length of the rigid frame member is about 160 mm, a length of the malleable section is about 50 mm, and a length of the active deflection section is about 22 mm.

5. An endoscope as in claim 2 where the malleable frame member is removably connected rigid frame member.

6. An endoscope as in claim 1 where the malleable section is adapted to be manually bent into a curved configuration, and where the distal active deflection section is configured to be bent by the at least one active deflection control wire without substantially movement of the malleable frame member, where the malleable frame member retains the malleable section in the curved configuration while the distal active deflection section is bent by the at least one active deflection control wire.

7. An endoscope as in claim 1 where the control section comprises a user actuated deflection control lever connected to the at least one active deflection control wire.

8. An endoscope as in claim 1 where the distal active deflection section comprises a plurality of the frame members at the distal active deflection section pivotably connected to each other.

9. An endoscope as in claim 1 where the frame member at the distal active deflection section comprises a tube shape with a plurality of slots therein.

10. An endoscope as in claim 1 where the malleable frame member comprises a metal tube.

11. An endoscope as in claim 1 where the malleable frame member comprises a super-elastic shape memory material.

12. An endoscope as in claim 1 further comprising at least one cable sheath covering the at least one active deflection control wire, where the at least one active deflection control wire extends through the active deflection section without being covered by the at least one cable sheath.

13. An endoscope as in claim 1 further comprising at least one cable sheath covering the at least one active deflection control wire, where the at least one cable sheath has a distal end which ends in the malleable frame member.

14. An endoscope as in claim 1 further comprising at least one cable sheath covering the at least one active deflection control wire, where the at least one cable sheath is directly fixedly connected to the malleable frame member.

15. A method comprising:
bending a malleable section of a shaft of an endoscope, where the shaft comprises an active deflection section located at a distal end of the shaft, where the active deflection section is connected by the malleable section to a control section of the endoscope, and where the active deflection section of the shaft is relocated when the malleable section is bent; and
deflecting the active deflection section to move the distal end of the endoscope shaft relative to the malleable section.

16. A method as in claim 15 where, deflecting the active deflection section comprises the control section being used to deflect the active deflection section, and where the malleable section is configured to substantially retain its shape and not be moved while the active deflection section is being deflected by the control section.

17. A method as in claim 15 where the malleable section is connected to the control section by an elongate rigid frame member, where bending the malleable section comprises the malleable section being bent relative to the elongate rigid frame member.

18. A method comprising:
connecting a frame member of an active deflection section of an endoscope shaft to a malleable frame member of the endoscope shaft, where the malleable frame member is part of a malleable deflectable section of the endoscope shaft; and
connecting the malleable frame member to a control section of an endoscope, where the malleable frame member connects the frame member of the active deflection section to the control section.

19. A method as in claim 18 where connecting the malleable frame member to the control section comprises connecting an elongate rigid frame member to the control section and connecting a rear end of the malleable frame member to a front end of the elongate rigid frame member.

20. A method as in claim 19 where connecting the malleable frame member to a front end of the elongate rigid frame member comprises removably connecting the malleable frame member to the rigid frame member.

21. A method as in claim 19 where connecting the malleable frame member to a front end of the elongate rigid frame member comprises permanently connecting the malleable frame member to the rigid frame member.

22. A method as in claim 21 where connecting the frame member of the active deflection section to the malleable frame member comprises permanently connecting the malleable frame member to the frame member of the active deflection section.

23. A method as in claim 15 further comprising locating at least one active deflection control wire through the malleable frame member from the control section to the active deflection section.
